(19) United States
(12) Patent Application Publication
(10) Pub. No.: US 2007/0212399 A1
(43) Pub. Date: Sep. 13, 2007

(54) Pyroligneous liquor sheet

(76) Inventor: Jung-Nam Kim, Seoul (KR)

Correspondence Address: JONES DAY, 222 EAST 41ST ST, NEW YORK, NY, 10017, US

(21) Appl. No.: 11/506,873

(22) Filed: Aug. 17, 2006

(30) Foreign Application Priority Data
Mar. 10, 2006 (KR) 10-2006-22755

(51) Int. Cl. A61K 9/70; A61K 36/09

(52) U.S. Cl. 424/443; 424/195.15; 424/725.1

(57) ABSTRACT

The present invention relates to a pyroligneous liquor sheet containing a pyroligneous powder, and more particularly to a pyroligneous liquor sheet having an excellent effect of removing waste matters by means of a contact surface with a human body. The pyroligneous liquor sheet according to the present invention provides a pyroligneous liquor sheet which is a pocket-type sack composed of an air-permeable sheet coming in contact with the skin and an air-impermeable sheet facing the air-permeable sheet and having a permeable network in at least one surface thereof, wherein a chamber, surrounded by the air-permeable sheet and the air-impermeable sheet, is filled with a pyroligneous powder mixture, wherein the pyroligneous powder mixture includes 0.05 to 1 part by weight of a tourmaline powder, based on 100 parts by weight of the pyroligneous powder prepared by mixing crystalline cellulose, starch, PCS (pregelatinized corn starch) with the pyroligneous liquor and granulating the resultant mixture, and wherein a plurality of oval holes are distributed over the air-permeable sheet, the oval holes having a horizontal length of 0.75 to 0.95 mm and a vertical length of 0.42 to 0.62 mm.

### BACKGROUND OF THE INVENTION

1. Field of the Invention

The present invention relates to a pyroligneous liquor sheet containing a pyroligneous powder, and more particularly to a pyroligneous liquor sheet having an excellent effect of removing waste matters on a contact surface with a human body.

2. Description of the Related Art

Pyroligneous liquor is referred to as a sap extracted when trees are carbonized, which is an aqueous solution obtained by condensing a white gas generated by means of thermal degradation. A mixed pyroligneous powder is prepared by grinding pyroligneous powder into fine particles and mixing the fine particles with starch, etc. The mixed pyroligneous powder was put into a sack made of nonwoven fabric materials, etc. to prepare a sheet-shaped pyroligneous sap powder sheet (hereinafter, referred to as “pyroligneous liquor sheet”), and then water poisons were removed from the pyroligneous liquor sheet for use. The pyroligneous liquor sheet has been used by attaching the pyroligneous liquor sheet to the sole of the foot on which spots spreading all over the body converge, and to various portions such as a knee, a shoulder and the like, if necessary.

Concerning such a pyroligneous liquor sheet, there haven many previous attempts to modify components of a pyroligneous liquor composition or improve a method for preparing the pyroligneous liquor composition, and also to improve a structure of the sheet.

For example, Korean Patent Publication No. 2001-14683, Korean Patent Publication No. 2003-58870, Korean Patent Publication No. 2003-60308, Korean Patent Publication No. 2003-71259, Korean Patent Publication No. 477351, Korean Patent Publication No. 2004-98788, Korean Patent Publication No. 2005-8414, Korean Patent Publication No. 2005-55302, Korean Patent Publication No. 2005-78736, Korean Patent Publication No. 2005-87214, Korean Utility Model Registration No. 376553, Japanese Patent Publication No. H11-302547, Japanese Patent Publication No. 2001-187889, etc. may be used as the reference.

FIG. 1 is a partially exploded schematic view showing a conventional pyroligneous liquor sheet. Referring to FIG. 1, the pyroligneous liquor sheet 10 is composed of an air-permeable sheet member 12 coming in contact with skin, an air-impermeable sheet member 14 facing the air-permeable sheet member 12, and a mixed pyroligneous powder 16 containing a pyroligneous powder filled into an internal space formed by attaching edges of the air-permeable sheet member 12 and the air-impermeable sheet member 14 to each other.

A plurality of through holes are formed in the air-permeable sheet member 12. As a result, the pyroligneous liquor sheet 10 has beneficial effects of removing off waste matters, purifying blood, restoring cells, etc. since the pyroligneous powder is in direct contact with a human body through the through holes if the pyroligneous liquor sheet 10 is attached onto the sole of the foot, etc.

If a size of the through holes is excessively large, particles of the pyroligneous powder are scattered through the through holes, while the effects of the pyroligneous liquor itself, for example removing waste matters, are remarkably deteriorated if a size of the through holes is excessively small.

Accordingly, it is very important to adjust the through holes of the pyroligneous liquor sheet to a suitable size.

Also, a plurality of through holes are substantially uniformly distributed over the pyroligneous liquor sheet as shown in FIG. 1. Accordingly, it is impossible to control the effects according to contact surfaces with a human body.

### SUMMARY OF THE INVENTION

Accordingly, the present invention is designed to solve the problems of the prior art, and therefore it is an object of the present invention to provide a shape and a size of a through hole capable of sufficiently preventing a pyroligneous powder, composed of fine particles, from being discharged out, and simultaneously maximizing an effect of removing waste matters.

Also, it is another object of the present invention to provide a pyroligneous liquor sheet capable of controlling removal efficiency and rate of waste matters by making a distribution of through holes different according to the portions coming in contact with a human body.

In order to accomplish the above objects, one aspect of the present invention provides a pyroligneous liquor sheet which is a pocket-type sack composed of an air-permeable sheet coming in contact with skin and an air-impermeable sheet facing the air-permeable sheet and having a permeable network in at least one surface thereof, wherein a chamber, surrounded by the air-permeable sheet and the air-impermeable sheet, is filled with a pyroligneous powder mixture, wherein the pyroligneous powder mixture includes 0.05 to 1 part by weight of a tourmaline powder, based on 100 parts by weight of the pyroligneous powder prepared by mixing crystalline cellulose, starch, PCS (pregelatinized corn starch) with the pyroligneous liquor and granulating the resultant mixture, and wherein a plurality of oval holes are distributed over the air-permeable sheet, the oval holes having a horizontal length of 0.75 to 0.95 mm and a vertical length of 0.42 to 0.62 mm.

In order to accomplish the above objects, another aspect of the present invention provides a pyroligneous liquor sheet which is a pocket-type sack composed of an air-permeable sheet coming in contact with skin and an air-impermeable sheet facing the air-permeable sheet and having a permeable network in at least one surface thereof, wherein a chamber, surrounded by the air-permeable sheet and the air-impermeable sheet, is filled with a pyroligneous powder mixture, wherein the pyroligneous powder mixture includes 0.05 to 5 part by weight of an Agaricus powder, based on 100 parts by weight of the pyroligneous powder prepared by mixing crystalline cellulose, starch, PCS (pregelatinized corn starch) with the pyroligneous liquor and granulating the resultant mixture, and wherein a plurality of oval holes are distributed over the air-permeable sheet, the oval holes having a horizontal length of 0.75 to 0.95 mm and a vertical length of 0.42 to 0.62 mm.

At this time, the oval holes may be uniformly or ununiformly distributed over a surface of the air-permeable sheet.

Especially, the air-permeable sheet may include a first region over which the oval holes are densely distributed and a second region over which the oval holes are coarsely distributed if the oval holes are ununiformly distributed over the air-permeable sheet. At this time, the first region is preferably arranged in a central area of the air-permeable sheet, and the second region is preferably arranged in a peripheral area of the air-permeable sheet surrounding the first region.

### BRIEF DESCRIPTION OF THE DRAWINGS

Other objects and aspects of the present invention will become apparent from the following description of embodiments with reference to the accompanying drawing in which:

FIG. 1 is a partially exploded schematic view showing a conventional pyroligneous liquor sheet.

FIG. 2 is a partially exploded schematic view showing a pyroligneous liquor sheet according to one preferred embodiment of the present invention.

FIG. 3 is a partially exploded schematic view showing a pyroligneous liquor sheet according to another preferred embodiment of the present invention.

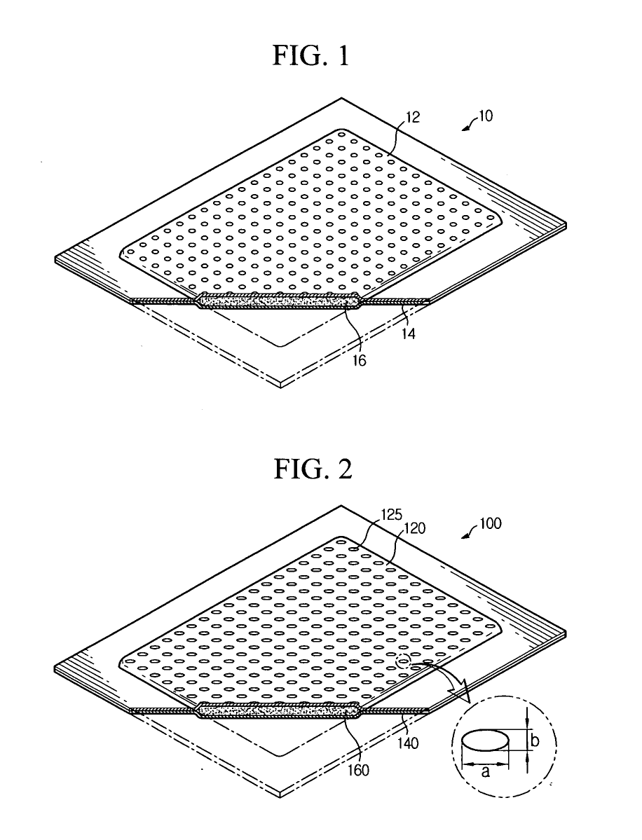

### DESCRIPTION OF THE PREFERRED EMBODIMENT

Hereinafter, preferred embodiments of the present invention will be described in detail referring to the accompanying drawings.

Firstly, FIG. 2 is a partially exploded schematic view showing a pyroligneous liquor sheet according to the present invention.

Referring to FIG. 2, the pyroligneous liquor sheet 100 according to the present invention is a pocket-type sack composed of an air-permeable sheet 120 coming in contact with skin and an air-impermeable sheet 140 facing the air-permeable sheet and having a permeable network in at least one surface thereof, wherein a chamber 130, surrounded by the air-permeable sheet 120 and the air-impermeable sheet 140, is filled with a pyroligneous powder mixture 160.

The pyroligneous powder mixture 160 includes 0.05 to 1 part by weight of tourmaline powder or Agaricus (Agaricus Blazei Murill or Panaeolus sp.) powder, based on 100 parts by weight of the pyroligneous powder prepared by mixing crystalline cellulose, starch, PCS (pregelatinized corn starch) with the pyroligneous liquor and granulating the resultant mixture. Also, maltodextrin and carboxymethyl cellulose sodium are preferably added to the mixture, if necessary.

The pyroligneous liquor is preferably, but not limited to, a pyroligneous liquor obtained by cooling a white gas exuding at a relatively initial stage when a latifoliate tree is boiled in a bath.

The pyroligneous powder is obtained by adding an equivalent amount of the pyroligneous liquor to crystalline cellulose, starch and PCS, followed by stirring and granulating the resultant mixture. At this time, the crystalline cellulose, the starch and the PCS are preferably mixed, for example, at a weight ratio of 5:2:3. Also, the pyroligneous powder preferably has a particle size of approximately 1.0 mm Φ.

Commercially available crystalline cellulose, starch and PCS may be used for the production method of the pyroligneous powder. The crystalline cellulose, the starch and the PCS are preferably mixed, for example, at a weight ratio of 5:2:3. And, the pyroligneous powder is produced by adding an equivalent amount of the pyroligneous liquor to the mixture and stirring the resultant mixture, followed by granulating the mixture into fine particles, preferably particles having a size of approximately 3.0 mm Φ using a granulator. At this time, a drying temperature is preferably approximately 50° C., and may be suitably varied according to the effect of a drying time. After drying, finely granulated powder having a particle size of approximately 1.0 mmΦ is preferably used as the pyroligneous powder.

A plurality of oval holes 125 are formed in the air-permeable sheet 120, as shown in FIG. 2. The oval holes 125 are substantially uniformly distributed over a surface of the air-permeable sheet 120.

The oval holes 125 have a horizontal length (a) of 0.75 to 0.95 mm and a vertical length (b) of 0.42 to 0.62 mm. Accordingly, an aqueous solution is easily passed through the oval hole 125, but the pyroligneous powder is not passed through the oval hole 125.

If the hole 125 is a round hole having a diameter of 0.75 to 0.95 mm, the pyroligneous powder is easily discharged out when the round hole is subject to external pressure. Also, air-permeability is deteriorated since the holes are closed by a pyroligneous powder paste kneaded with water if a horizontal length or a vertical length of the hole is not greater than 0.75 mm or 0.42 mm, respectively, while the pyroligneous powder particles are discharged out if a horizontal length or a vertical length of the hole exceeds 0.95 mm or 0.62 mm, respectively.

Accordingly, the holes, formed in the surface of the air-permeable sheet according to the present invention, should be limited to an oval hole having the above-mentioned range of the horizontal length (0.75 to 0.95 mm) and the vertical length (0.42 to 0.62 mm)

Materials of the air-permeable sheet and the air-impermeable sheet are not particularly limited, but preferably a nonwoven fabric in an aspect of its durability.

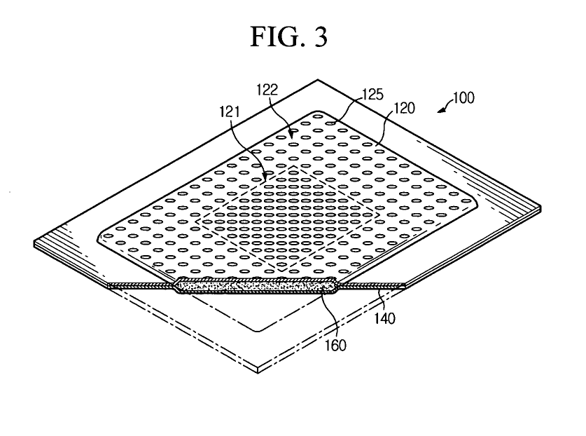

Also, FIG. 3 is a partially exploded schematic view showing a pyroligneous liquor sheet according to another embodiment of the present invention.

The pyroligneous liquor sheet of FIG. 3 has the nearly same configuration as in FIG. 2. However, the distribution of the oval holes formed in the surface of the air-permeable sheet is different from the distribution as shown in FIG. 2.

In the case of the pyroligneous liquor sheet as shown in FIG. 2, the oval holes 125 are substantially uniformly distributed over the surface of the air-permeable sheet 120, but the oval holes 125 are ununiformly distributed over the surface of the air-permeable sheet 120 in the pyroligneous liquor sheet as show in FIG. 3.

That is to say, the pyroligneous liquor sheet 120 of FIG. 3 includes a first region 121 over which the oval holes are densely distributed and a second region 122 over which the oval holes are coarsely distributed.

The first region 121 is preferably arranged in a central area of the air-permeable sheet, and the second region 122 is preferably arranged in a peripheral area of the air-permeable sheet surrounding the first region 121.

Generally, if the pyroligneous liquor sheet according to the present invention is attached to the sole of a human foot, the central area of the air-permeable sheet is closely attached to the skin, while the peripheral area of the air-permeable sheet is detached from the skin. Also, the pyroligneous powder mixture tends to converge in the central area of the sheet.

Accordingly, the air-permeable holes 125 are preferably densely distributed in the first region 121 having a large contact area with the skin, and the air-permeable holes 125 are preferably coarsely distributed in the second region 122 having a small contact area with the skin.

If the air-permeable holes 125 are uniformly distributed over the sheet as shown in FIG. 2, a possibility of discharging the pyroligneous powder through the holes is increased without an improved effect of removing waste matters. Accordingly, the air-permeable holes are preferably differently distributed in consideration of the contact area with skin in order to maximally inhibit the pyroligneous powder from being discharged out and simultaneously improve an air-permeability on the skin, as shown in FIG. 3.

That is to say, a large number of oval holes are preferably distributed in a region coming often in contact with the skin (the first region 121) in consideration of the air-permeability, and a small number of oval holes are preferably distributed in a region failing to come in contact with the skin (the second region 122) in consideration of an outflow of the pyroligneous powder.

The second region 122 has approximately 40 to 70% air-permeable holes of the air-permeable holes arranged in the first region 121. An air-permeability is deterioarated if the number of the air-permeable holes formed in the second region 122 is 40% or less of that of the first region 121, while a possibility of discharging the pyroligneous powder is increased if the number of the air-permeable holes exceeds 70% of that of the first region 121.

### Embodiment 1

1. Production of Pyroligneous Powder

Firstly, 12.0 kg of crystalline cellulose, 4.8 kg of starch, 7.2 kg of PCS were mixed. 20 L of pyroligneous liquor was added to the mixture, and stirred to obtain particles having a particle size of approximately 3.0 mm Φ, and then the particles were dried at approximately 50° C. The dried particles were ground into fine powder having a size of 1.0 mm Φ, which was used as the pyroligneous powder.

2. Production of Tourmaline-Containing Pyroligneous Powder

10 kg of the pyroligneous powder prepared in the step 1, 80 kg of maltodextrin, 10 kg of carboxymethyl cellulose sodium and 0.6 kg of tourmaline were mixed thsubstantially to obtain a tourmaline-containing pyroligneous powder.

3. Production of Tourmaline-Containing Pyroligneous Powder Sack

The tourmaline-containing pyroligneous powder prepared in the step 2 was divided into an amount of 4 g, and the tourmaline-containing pyroligneous powder mixture was packed in a sack.

At this time, the used sack is made of a nonwoven fabric having a configuration as shown in FIG. 2 or FIG. 3.

### Embodiment 2

1. Production of Pyroligneous Powder

Firstly, 12.0 kg of crystalline cellulose, 4.8 kg of starch, 7.2 kg of PCS were mixed. 20 L of pyroligneous liquor was added to the mixture, and stirred to obtain particles having a particle size of approximately 3.0 mm Φ, and then the particles were dried at approximately 50° C. The dried particles were ground into fine powder having a size of 1.0 mm Φ, which was used as the pyroligneous powder.

2. Production of Agaricus Powder-Containing Pyroligneous Powder

10 kg of the pyroligneous powder prepared in the step 1, 80 kg of maltodextrin, 10 kg of carboxymethyl cellulose sodium and 0.6 kg of Agaricus powder were mixed thsubstantially to obtain an Agaricus powder-containing pyroligneous powder.

3. Production of Agaricus Powder-Containing Pyroligneous Powder Sack

The Agaricus powder-containing pyroligneous powder prepared in the step 2 was divided into an amount of 4 g, and the Agaricus powder-containing pyroligneous powder mixture was packed in a sack.

At this time, the used sack is made of a nonwoven fabric having a configuration as shown in FIG. 2 or FIG. 3.

Also, the sack was attached and fixed to an adhesive sheet, and then the sack and the adhesive sheet were covered together with a cover of the adhesive sheet. The pyroligneous liquor sheet was packed in a vinyl bag, wherein a separate moisture absorbent was sealed in the sack so as to prevent the sack from absorbing moisture in the adhesive sheet.

It should be understood that the terms used in the specification and appended claims should not be construed as limited to general and dictionary meanings, but interpreted based on the meanings and concepts corresponding to technical aspects of the present invention on the basis of the principle that the inventor is allowed to define terms appropriately for the best explanation.

Therefore, the description proposed herein is just a preferable example for the purpose of illustrations only, not intended to limit the scope of the invention, so it should be understood that other equivalents and modifications could be made thereto without departing from the spirit and scope of the invention.

It should be understood that the detailed description and specific examples, while indicating preferred embodiments of the invention, are given by way of illustration only, since various changes and modifications within the spirit and scope of the invention will become apparent to those skilled in the art from this detailed description.

### APPLICABILITY TO THE INDUSTRY

The pyroligneous liquor sheet according to the present invention was designed to ensure a maximum moisture permeability upon contact to the skin and inhibit a fine pyroligneous powder from leaking out.

Accordingly, The pyroligneous liquor sheet according to the present invention may be useful to maximally inhibit a pyroligneous powder from leaking out upon its use, as well as maximize a formation effect on a human body, or an effect of removing waste matters.

What is claimed is:

1. A pyroligneous liquor sheet which is a pocket-type sack composed of an air-permeable sheet coming in contact with skin and an air-impermeable sheet facing the air-permeable sheet and having a permeable network in at least one surface thereof,
wherein a chamber, surrounded by the air-permeable sheet and the air-impermeable sheet, is filled with a pyroligneous powder mixture,
wherein the pyroligneous powder mixture includes 0.05 to 1 part by weight of a tourmaline powder, based on 100 parts by weight of the pyroligneous powder prepared by mixing crystalline cellulose, starch, PCS (pregelatinized corn starch) with the pyroligneous liquor and granulating the resultant mixture, and
wherein a plurality of oval holes are distributed over the air-permeable sheet, the oval holes having a horizontal length of 0.75 to 0.95 mm and a vertical length of 0.42 to 0.62 mm.

2. The pyroligneous liquor sheet according to claim 1,
wherein the oval holes are uniformly distributed over a surface of the air-permeable sheet.

3. The pyroligneous liquor sheet according to claim 1,
wherein the air-permeable sheet includes a first region over which the oval holes are densely distributed and a second region over which the oval holes are coarsely distributed.

4. The pyroligneous liquor sheet according to claim 3,
wherein the first region is arranged in a central area of the air-permeable sheet, and the second region is arranged in a peripheral area of the air-permeable sheet surrounding the first region.

5. The pyroligneous liquor sheet according to claim 4,
wherein the second region has 40 to 70% oval holes of the first region.

6. A pyroligneous liquor sheet which is a pocket-type sack composed of an air-permeable sheet coming in contact with skin and an air-impermeable sheet facing the air-permeable sheet and having a permeable network in at least one surface thereof,
wherein a chamber, surrounded by the air-permeable sheet and the air-impermeable sheet, is filled with a pyroligneous powder mixture,
wherein the pyroligneous powder mixture includes 0.05 to 5 part by weight of an Agaricus powder, based on 100 parts by weight of the pyroligneous powder prepared by mixing crystalline cellulose, starch, PCS (pregelatinized corn starch) with the pyrolignebus liquor and granulating the resultant mixture, and
wherein a plurality of oval holes are distributed over the air-permeable sheet, the oval holes having a horizontal length of 0.75 to 0.95 mm and a vertical length of 0.42 to 0.62 mm.

7. The pyroligneous liquor sheet according to claim 6,
wherein the oval holes are uniformly distributed over a surface of the air-permeable sheet.

8. The pyroligneous liquor sheet according to claim 6,
wherein the air-permeable sheet includes a first region over which the oval holes are densely distributed and a second region over which the oval holes are coarsely distributed.

9. The pyroligneous liquor sheet according to claim 8,
wherein the first region is arranged in a central area of the air-permeable sheet, and the second region is arranged in a peripheral area of the air-permeable sheet surrounding the first region.

10. The pyroligneous liquor sheet according to claim 9,
wherein the second region has 40 to 70% oval holes of the first region.
